SEQUENCE LISTING (7 sequences)

SEQ ID NO: 1 - DNA, 24 bp, synthetic construct
  source 1..24: mol_type = other DNA
ttattattattatcacaagttagg

SEQ ID NO: 2 - RNA, 22 nt, synthetic construct
  source 1..22: mol_type = other RNA
tccctgagaccctaacttgtga

SEQ ID NO: 3 - DNA, 42 bp, synthetic construct
  source 1..42: mol_type = other DNA
ggccgtcaacatcagtctgataagctaaacatgatgacggcc

SEQ ID NO: 4 - RNA, 22 nt, synthetic construct
  source 1..22: mol_type = other RNA
tagcttatcagactgatgttga

SEQ ID NO: 5 - DNA, 17 bp, synthetic construct
  source 1..17: mol_type = other DNA
tttttggccgtcatcat

SEQ ID NO: 6 - DNA, 27 bp, synthetic construct
  source 1..27: mol_type = other DNA
tactcccccaggtgcatgatgacggcc

SEQ ID NO: 7 - DNA, 27 bp, synthetic construct
  source 1..27: mol_type = other DNA
gcacctgggggagtaggccgtcatcat

(19) United States
(12) Patent Application Publication
(10) Pub. No.: US 2025/0346944 A1
(43) Pub. Date: Nov. 13, 2025

(54) ANTIMONENE-BASED SURFACE PLASMON RESONANCE PRISM COUPLER SENSOR, MIRNA DETECTION DEVICE, DUAL-RETARDER POLARIMETRY SYSTEM AND METHOD FOR DETECTING CONCENTRATION OF MIRNA IN BIOLOGICAL SAMPLE

(71) Applicant: NATIONAL UNITED UNIVERSITY, MIAOLI (TW)

(72) Inventors: Quoc-Hung Phan, MIAOLI (TW); Quoc-Thinh Dinh, MIAOLI (TW)

(73) Assignee: NATIONAL UNITED UNIVERSITY, MIAOLI (TW)

(21) Appl. No.: 18/768,708

(22) Filed: Jul. 10, 2024

(30) Foreign Application Priority Data
May 9, 2024 (TW) 113117202

(51) Int. Cl. C12Q 1/6825; C12Q 1/6876; G01N 21/59

(52) U.S. Cl. CPC C12Q 1/6825; C12Q 1/6876; G01N 21/59; C12Q 2600/178; G01N 2021/5903

(57) ABSTRACT

An antimonene-based surface plasmon resonance prism coupler sensor, a miRNA detection device, a dual-retarder polarimetry system, and a method for detecting a concentration of a miRNA in a biological sample are provided. The antimonene-based surface plasmon resonance prism coupler sensor includes a prism, a tantalum pentoxide thin film layer, a gold thin film layer and an antimonene layer in sequence from bottom to top. The miRNA detection device includes the antimonene-based surface plasmon resonance prism coupler sensor, a capture nucleic acid, a detection probe and a reporter nucleic acid set. The dual-retarder polarimetry system includes the miRNA detection device, a light source, a polarizer, a liquid crystal phase variable retarder group, a Stokes polarimeter and a computing module. The method includes using the dual-retarder polarimetry system with a decomposition Mueller matrix to detect the concentration of the miRNA in the biological sample.

### RELATED APPLICATIONS

This application claims priority to Taiwan Application Serial Number 113117202, filed May 9, 2024, which is herein incorporated by reference.

### SEQUENCE LISTING XML

The Sequence Listing XML associated with this application is filed in electronic format via EFS-Web and is hereby incorporated by reference into the specification in its entirety. The name of the XML file containing the Sequence Listing is CP-6562-US_SEQ_LIST. The size of the XML file is 7.31 KB, and the XML file was created on Jun. 25, 2024.

### BACKGROUND

### Technical Field

The present disclosure relates to a surface plasmon resonance sensor and use thereof. More particularly, the present disclosure relates to an antimonene-based surface plasmon resonance prism coupler sensor, a miRNA detection device, a dual-retarder polarimetry system and a method for detecting a miRNA in a biological sample.

### Description of Related Art

MicroRNA (miRNA) is a highly conserved non-coding RNA with a length of approximately 18-25 nucleotides, which can regulate the gene expression of organisms. In recent years, research results have shown that the expression of miRNA in cancer patients can be used as a tool for cancer diagnosis and prognosis. The expression of miRNA in cancer patients can even further predict the survival rate of cancer patients, and has become a new generation of cancer biomarkers. Therefore, materials and methods for detection and quantification of miRNA are important and critical.

In order to make cancer detection a part of daily life, there are currently many methods for cancer diagnostic detection, including sensing systems based on electrochemical, optical and nanoparticle-based technologies. These biosensors provide many advantages, including exceptional sensitivity, a rapid response time, and the possibility of miniaturization and incorporation into point-of-care (POC) instruments. However, the accuracy and resolution of the above-proposed methods are not high enough for practical miRNA detection applications, and the equipment structure and calibration process of existing methods are complex and expensive. Therefore, simple, reliable, and efficient miRNA detection methods are still needed.

### SUMMARY

According to one embodiment of the present disclosure, an antimonene-based surface plasmon resonance prism coupler sensor includes a prism, a tantalum pentoxide thin film layer, a gold thin film layer and an antimonene layer. The prism has a surface. The tantalum pentoxide thin film layer is disposed on the surface of the prism. The gold thin film layer is disposed on the tantalum pentoxide thin film layer. The antimonene layer is disposed on the gold thin film layer.

According to another embodiment of the present disclosure, a miRNA detection device, which is for detecting a miRNA in a biological sample, includes the aforementioned antimonene-based surface plasmon resonance prism coupler sensor, a capture nucleic acid, a detection probe and a reporter nucleic acid set. The capture nucleic acid is seeded on the antimonene layer of the antimonene-based surface plasmon resonance prism coupler sensor, and the capture nucleic acid specifically binds to the miRNA. The detection probe is for amplifying detection signals, and the detection probe includes a gold nanoparticle, a first assistant nucleic acid and a second assistant nucleic acid. Bases at one end of the first assistant nucleic acid and bases at one end of the second assistant nucleic acid are respectively connected to the gold nanoparticle, and the first assistant nucleic acid specifically binds to the capture nucleic acid. The reporter nucleic acid set includes a first reporter nucleic acid and a second reporter nucleic acid, in which the first reporter nucleic acid specifically binds to the capture nucleic acid, and the second reporter nucleic acid specifically binds to the capture nucleic acid and the first assistant nucleic acid.

According to one another embodiment of the present disclosure, a dual-retarder polarimetry system includes the aforementioned miRNA detection device, a light source, a polarizer, a liquid crystal phase variable retarder group, a Stokes polarimeter and a computing module. The miRNA detection device is in contact with a biological sample. The light source is configured to generate an incident light, and the incident light is incident on the miRNA detection device along a light path. The polarizer is disposed between the light source and the miRNA detection device, and is configured to polarize the incident light into a polarized light. The liquid crystal phase variable retarder group is disposed between the polarizer and the miRNA detection device, and is configured to receive the polarized light and change polarization states of the polarized light to form a modulated polarized light. The liquid crystal phase variable retarder group includes a first liquid crystal phase variable retarder with an angle of a main axis at 90° and a second liquid crystal phase variable retarder with an angle of the main axis at 45°. The second liquid crystal phase variable retarder is closer to the miRNA detection device than the first liquid crystal phase variable retarder. The Stokes polarimeter is configured to receive a reflected light formed by the modulated polarized light after passing through the miRNA detection device and generate optical information. The computing module is configured to receive the optical information generated by the Stokes polarimeter, calculate a decomposition Muller matrix corresponding, and then calculate a linear birefringence property and a circular dichroism property of the biological sample, so as to detect a concentration of a miRNA in the biological sample.

According to still another embodiment of the present disclosure, a method for detecting a concentration of a miRNA in a biological sample includes steps as follows. The aforementioned dual-retarder polarimetry system is provided. The biological sample is mixed with the detection probe to form a mixture. The mixture and the reporter nucleic acid set are added to the antimonene-based surface plasmon resonance prism coupler sensor, in which the capture nucleic acid binds to the miRNA in the biological sample and forms a complex with the detection probe and the reporter nucleic acid set. The light source is turned on to generate an incident light and incident on the complex along a light path. An optical information acquisition step is performed, in which the Stokes polarimeter receives a reflected light formed after passing through the complex and generates optical information. A calculation step is performed, in which the computing module receives the optical information generated by the Stokes polarimeter, calculates a decomposition Muller matrix corresponding, and then calculates a linear birefringence property and a circular dichroism property of the biological sample, so as to detect the concentration of the miRNA in the biological sample.

### BRIEF DESCRIPTION OF THE DRAWINGS

The present disclosure can be more fully understood by reading the following detailed description of the embodiment, with reference made to the accompanying drawings as follows:

FIG. 1 is a schematic view of an antimonene-based surface plasmon resonance prism coupler sensor according to one embodiment of the present disclosure.

FIG. 2A and FIG. 2B are analysis results of sensitivity of the antimonene-based surface plasmon resonance prism coupler sensor of Example 1.

FIG. 2C is analysis result of electric field enhancement distribution of the antimonene-based surface plasmon resonance prism coupler sensor of Example 1.

FIG. 3A is a schematic view of a miRNA detection device according to another embodiment of the present disclosure.

FIG. 3B is an assembly schematic view of the miRNA detection device in FIG. 3A.

FIG. 4 is a schematic view of a dual-retarder polarimetry system according to one another embodiment of the present disclosure.

FIG. 5 is a process schematic view showing the amplification of detection signals by the miRNA detection device according to still another embodiment of the present disclosure.

FIG. 6A shows a relationship between a principal angle of a fast axis of a linear birefringence property of miRNA (miR-125 and miR-21) and a concentration of miRNA.

FIG. 6B shows a relationship between a rotation angle of a circular dichroism property of miRNA (miR-125 and miR-21) and a concentration of miRNA.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

### [Antimonene-Based Surface Plasmon Resonance Prism Coupler Sensor]

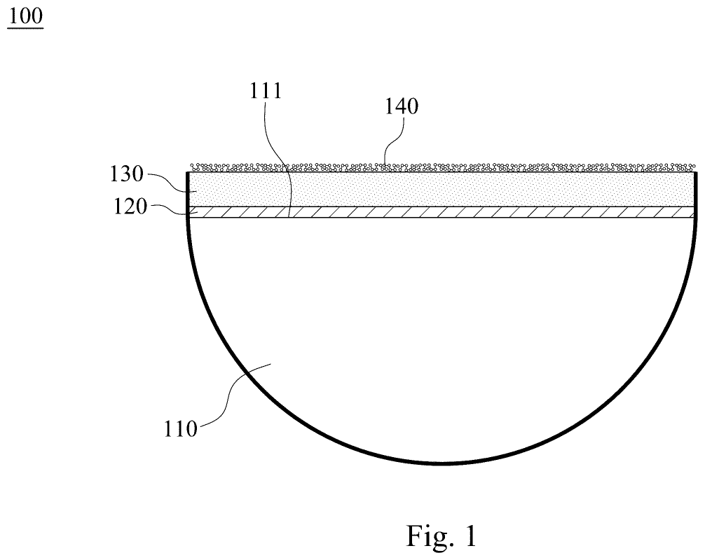

Reference is made to FIG. 1, which is a schematic view of an antimonene-based surface plasmon resonance prism coupler sensor 100 according to one embodiment of the present disclosure. The antimonene-based surface plasmon resonance prism coupler sensor 100 includes a prism 110, a tantalum pentoxide thin film layer 120, a gold thin film layer 130 and an antimonene layer 140. The prism 110 has a surface 111. The tantalum pentoxide thin film layer 120 is disposed on the surface 111 of the prism 110. The gold thin film layer 130 is disposed on the tantalum pentoxide thin film layer 120. The antimonene layer 140 is disposed on the gold thin film layer 130.

In some embodiments, the prism 110 can be a half-ball glass lens. A thickness of the gold thin film layer 130 can be greater than a thickness of the tantalum pentoxide thin film layer 120 and a thickness of the antimonene layer 140. The antimonene layer 140 can be composed of a plurality of layers of antimonene. Antimonene has a sp² bonded honeycomb crystal lattice and exhibits strong spin-orbit coupling, great stability and hydrophilicity. The physical and chemical properties of antimonene are significantly better than typical two-dimensional materials such as graphene, MoS₂ and black phosphorus. The performance of surface plasmon resonance sensor can be enhanced through high surface area ratio, high carrier mobility, high stability, and biomolecule compatibility of antimonene.

In the antimonene-based surface plasmon resonance prism coupler sensor 100 of Example 1 of the present disclosure (hereinafter referred to as Example 1), the prism 110 is a half-ball lens (BK7, Thorlabs ACL1210U), and the surface 111 of the prism 110 is coated with one tantalum pentoxide thin film layer 120 (Ta₂O₅), one gold thin film layer 130 (Au) and several antimonene layers 140.

Antimonene in Example 1 was synthesized using liquid phase exfoliation (LPE) technique, including the use of isopropanol (IPA) method and N-methyl-2-pyrrolidone (NMP) method. The process involved the exfoliation the bulk antimony crystals (7440360, Thermoscientific) into a stable suspension of micrometer-sized layered antimonene through the use of 50 mL of a mixture of IPA/H₂O (ratio of 4:1) or 50 mL of a mixture of NMP/H₂O (ratio of 4:1) without the assistance of surfactant. The antimony crystal was sonicated for 40 minutes at a frequency of 24 kHz, a power of 400 W, and a temperature of 30° C. To eliminate any unexfoliated antimony, the suspension was then subjected to centrifugation at 3,000 rpm for 3 minutes at 30° C., yielding the final layered nanosheet. When coating the antimonene layer, 4 g of poly(methyl methacrylate) (PMMA) and 100 mL of anisole were sonicated for 40 minutes at a frequency of 24 kHz, a power of 400 W, and a temperature of 30° C. The resist solution was then spin-coated on the flat surface of BK7 (hereinafter referred to as SPR) coated with the tantalum pentoxide thin film layer 120 and the gold thin film layer 130 at a speed of 600 rpm for 6 seconds, and then spin-coated at a speed of 4000 rpm for 30 seconds. The SPR was rinsed with de-ionized water 3 times for 10 minutes each time. After completion, the SPR was dried at room temperature for 30 minutes, further dried in a microwave at 100° C. for 20 minutes, soaked in acetone 3 times to remove residual traces of PMMA, and finally dried once again in a microwave at 50° C. for 10 minutes to obtain Example 1.

The radius of the prism 110 of Example 1 is 10 mm, and the refractive index is n_{BK7}=1.5168. The thickness of the tantalum pentoxide thin film layer 120 is 10 nm, and the refractive index is n_{Ta2O5}=2.1203+0.00099i. The thickness of the gold thin film layer 130 is 40 nm, and the refractive index is n_{Au}=0.36-2.9i. The thickness of the antimonene layer 140 is 3 nm, and the refractive index is n₄=2.1+0.45i.

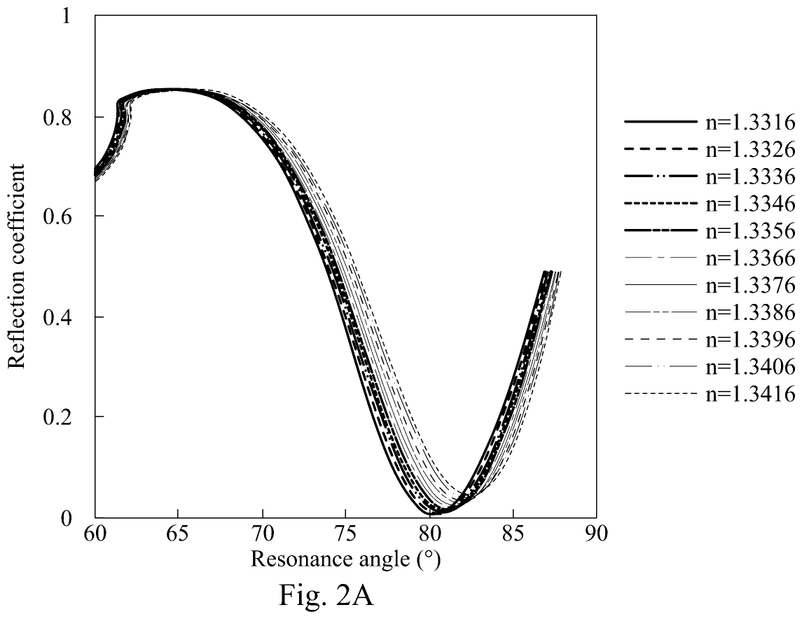

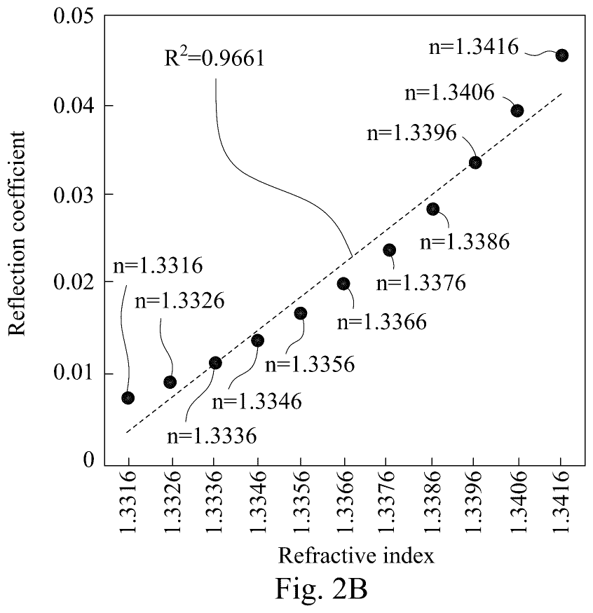

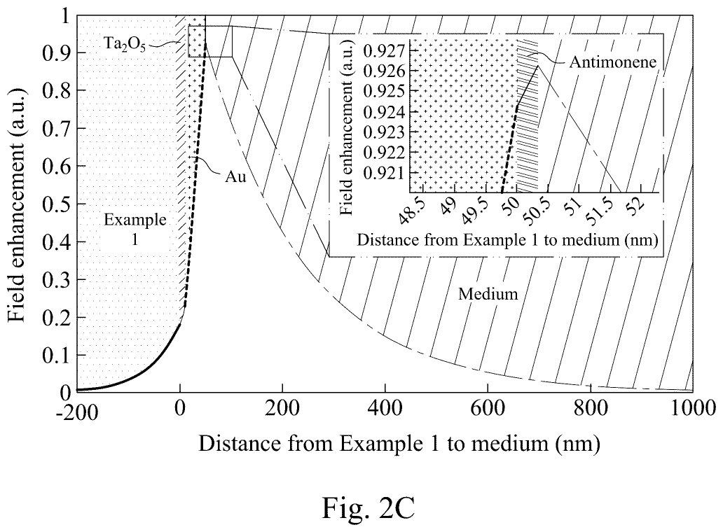

Reference is made to FIG. 2A and FIG. 2B, which are analysis results of sensitivity of Example 1. The results in FIG. 2A show that the resonance angle of Example 1 is approximately 80° and the minimum reflection coefficient is 0.01. The reflection coefficient of Example 1 is calculated using a multi-layer mathematical model. The results in FIG. 2B show that the reflection coefficient of Example 1 is linearly related to the refractive index, and a correlation coefficient is R²=0.9661.

Reference is made to FIG. 2C, which is analysis result of electric field enhancement distribution of Example 1. The result shows that the BK7 glass material provides total internal reflection. Moreover, the tantalum pentoxide thin film layer 120 with anisotropy has a smaller thickness and higher refractive index and acts as a waveguide, thereby enhancing the electric field at the analyte interface. The gold thin film layer 130 with isotropy induces a strong surface plasmon excitation effect. Finally, the antimonene layer 140 enhances the electric field from 0.924 (a.u.) to 0.926 (a.u.), with a maximum peak observed at the interface between the antimonene layer 140 and the medium for sensing.

[miRNA Detection Device]

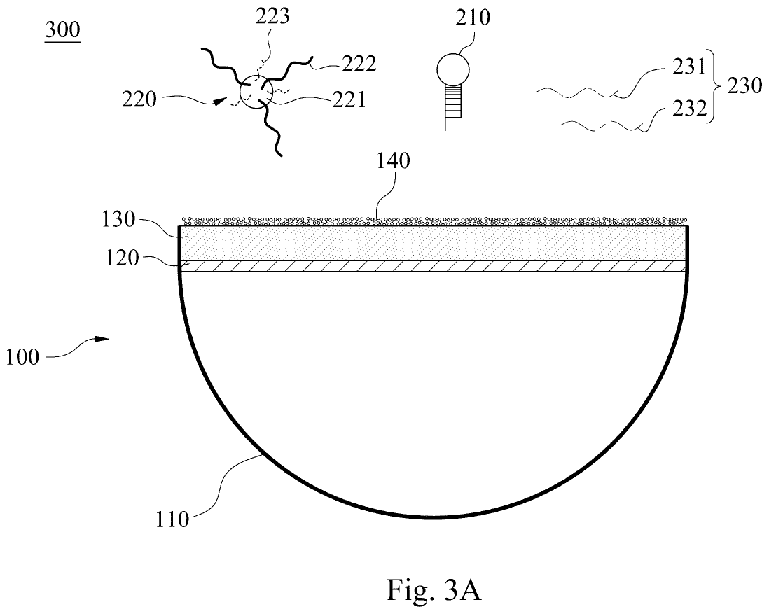

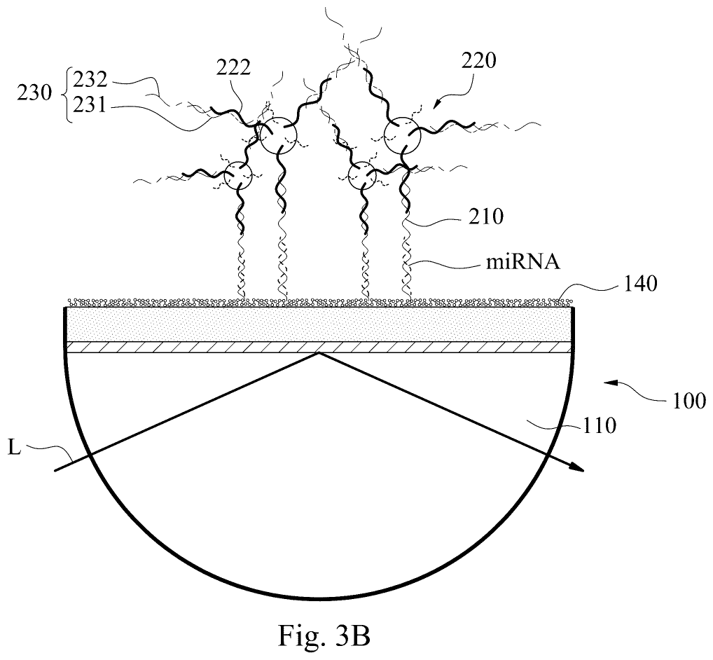

Reference is made to FIG. 3A and FIG. 3B. FIG. 3A is a schematic view of a miRNA detection device 300 according to another embodiment of the present disclosure, and FIG. 3B is an assembly schematic view of the miRNA detection device 300 in FIG. 3A. The miRNA detection device 300 is for detecting a miRNA in a biological sample.

As shown in FIG. 3A, the miRNA detection device 300 includes the aforementioned antimonene-based surface plasmon resonance prism coupler sensor 100, a capture nucleic acid 210, a detection probe 220 and a reporter nucleic acid set 230. The detection probe 220 is for amplifying detection signals, which includes a gold nanoparticle 221, a first assistant nucleic acid 222 and a second assistant nucleic acid 223. The reporter nucleic acid set 230 includes a first reporter nucleic acid 231 and a second reporter nucleic acid 232. As shown in FIG. 3A and FIG. 3B, the capture nucleic acid 210 is seeded on the antimonene layer 140 of the antimonene-based surface plasmon resonance prism coupler sensor 100, and the capture nucleic acid 210 specifically binds to the miRNA to be detected. Bases at one end of the first assistant nucleic acid 222 and bases at one end of the second assistant nucleic acid 223 of the detection probe 220 are respectively connected to the gold nanoparticle 221, and the first assistant nucleic acid 222 specifically binds to the capture nucleic acid 210. The first reporter nucleic acid 231 of the reporter nucleic acid set 230 specifically binds to the capture nucleic acid 210, and the second reporter nucleic acid 232 specifically binds to the capture nucleic acid 210 and the first assistant nucleic acid 222. In the miRNA detection device 300, total internal reflection occurs when an incident light L is incident on the antimonene-based surface plasmon resonance prism coupler sensor 100.

### [Dual-Retarder Polarimetry System]

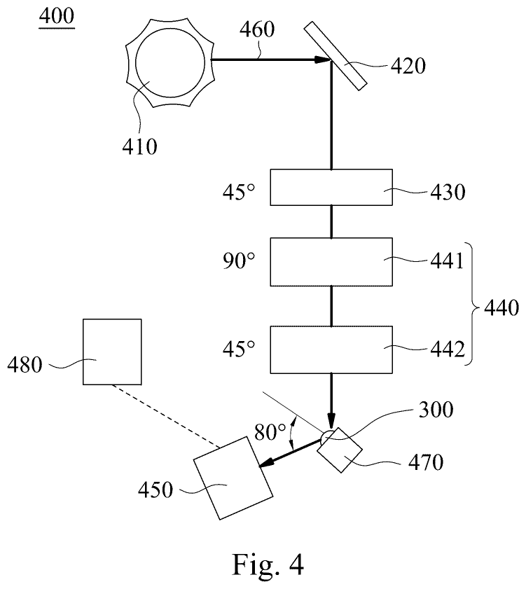

Reference is made to FIG. 4, which is a schematic view of a dual-retarder polarimetry system 400 according to one another embodiment of the present disclosure. The dual-retarder polarimetry system 400 includes the aforementioned miRNA detection device 300, a light source 410, a polarizer 430, a liquid crystal phase variable retarder group 440, a Stokes polarimeter 450 and a computing module 480.

The miRNA detection device 300 is in contact with a biological sample. The light source 410 is configured to generate an incident light, and the incident light is incident on the miRNA detection device 300 along a light path 460. The polarizer 430 is disposed between the light source 410 and the miRNA detection device 300, and is configured to polarize the incident light into a polarized light. In addition, the dual-retarder polarimetry system 400 can further include a light path adjustment element 420. The light path adjustment element 420 is disposed between the light source 410 and the polarizer 430, and the light path adjustment element 420 is configured to adjust a light path direction of the incident light. The light path adjustment element 420 can be a reflecting mirror, a refracting mirror, a beam splitter, a prism or a combination thereof.

The liquid crystal phase variable retarder group 440 is disposed between the polarizer 430 and the miRNA detection device 300, and is configured to receive the polarized light and change polarization states of the polarized light to form a modulated polarized light. The liquid crystal phase variable retarder group 440 includes a first liquid crystal phase variable retarder 441 with an angle of a main axis at 90° and a second liquid crystal phase variable retarder 442 with an angle of the main axis at 45°, in which the second liquid crystal phase variable retarder 442 is closer to the miRNA detection device 300 than the first liquid crystal phase variable retarder 441. The Stokes polarimeter 450 is configured to receive a reflected light formed by the modulated polarized light after passing through the miRNA detection device 300 and generate optical information.

The computing module 480 is configured to receive the optical information generated by the Stokes polarimeter 450, calculate a decomposition Muller matrix corresponding, and then calculate a linear birefringence property and a circular dichroism property of the biological sample, so as to detect a concentration of a miRNA in the biological sample.

In addition, the dual-retarder polarimetry system 400 can further include a cuvette 470, which is connected to the miRNA detection device 300 and used to store the biological sample. The cuvette 470 has a hole (not shown) to allow direct contact between the biological sample and the miRNA detection device 300 to avoid optical interference in the cuvette 470.

In detail, the antimonene-based surface plasmon resonance prism coupler sensor 100 in the miRNA detection device 300 is used to generate total internal reflection in the dual-retarder polarimetry system 400 and cooperates with the decomposition Mueller matrix to calculate the linear birefringence property and the circular dichroism property of the biological sample to detect the concentration of miRNA in the biological sample. During measurement, the biological sample is injected into the cuvette 470. The Stokes vector of the polarized light emitted by the polarizer 430 and the liquid crystal phase variable retarder group 440 in the dual-retarder polarimetry system 400 can be expressed as the following equation (4):

$$S = {[ 1 - sin {δ}_{1} sin {δ}_{2} cos {δ}_{1} sin {δ}_{1} cos {δ}_{2} ]}^{⊤} ; \qquad ( 4 )$$

where δ₁ and δ₂ are the adjustable phase retardations of the first liquid crystal phase variable retarder 441 and the second liquid crystal phase variable retarder 442 respectively, and S is a Stokes vector of the reflected light. In the present disclosure, four modulated polarization states can be generated through the combination of the first liquid crystal phase variable retarder 441 and the second liquid crystal phase variable retarder 442, including 3 linear polarization states (angles of a main axis are 0°, 45° and 90°) and 1 right-handed circular polarization state (R).

The relationship between the Stokes vector and a Muller matrix of the biological sample can be expressed as equation (5):

$$S = {MS}^{′} ; \qquad ( 5 )$$

where M is the Muller matrix, S is the Stokes vector of the reflected light, and S′ is a Stokes vector of the incident light.

The decomposition Muller matrix used to detect the linear birefringence property and the circular dichroism property of the biological sample is expressed as equation (1):

$${M}_{sample} = {M}_{l b} {M}_{c d} {M}_{R} {M}_{D} ; \qquad ( 1 )$$

where Mₛₐₘₚₗₑ is a Muller matrix of the biological sample, M_{lb} is a Muller matrix of the linear birefringence property of the biological sample, M_{cd} is a Muller matrix of the circular dichroism property of the biological sample, M_{R} is a Mueller matrix of a reflectance of the miRNA detection device 300, and M_{D} is a Mueller matrix of a depolarization effect of the biological sample.

The decomposition Muller matrix can be expanded as equation (6) and equation (7):

$${[ S ]}_{0 ° , 45 ° , 90 ° , R} = {M}_{l b} {M}_{c d} {M}_{D} {{M}_{R} [ {S}^{′} ]}_{0 ° , 45 ° , 90 ° , R} ; \qquad ( 6 )$$

$${M}_{l b} {M}_{c d} {M}_{D} {{M}_{R} [ {S}^{′} ]}_{0 ° , 45 ° , 90 ° , R} = {[ \begin{matrix}{M}_{11} & {M}_{12} & {M}_{13} & {M}_{14} \\ {M}_{21} & {M}_{22} & {M}_{23} & {M}_{24} \\ {M}_{31} & {M}_{32} & {M}_{33} & {M}_{34} \\ {M}_{41} & {M}_{42} & {M}_{34} & {M}_{44}\end{matrix} ] [ \begin{matrix}{S}_{0}^{′} \\ {S}_{1}^{′} \\ {S}_{2}^{′} \\ {S}_{3}^{′}\end{matrix} ]}_{0 ° , 45 ° , 90 ° , R} ; \qquad ( 7 )$$

where [S]_{0°,45°,90°,R} is the Stokes vector of the reflected light in the linear polarization state with angles of the main axis at 0°, 45°, 90° and the right-handed circular polarization state, [S′]_{0°,45°,90°,R} is the Stokes vector of the incident light in the linear polarization state with angles of the main axis at 0°, 45°, 90° and the right-handed circular polarization state, Mij is an element of the Mueller matrix, and S₀′, S₁′, S₂′ and S₃′ are Stokes parameters of the Stokes vector of the incident light.

The linear birefringence property is calculated as a principal angle of a fast axis (α), the circular dichroism property is calculated as a rotation angle (R), and the principal angle of the fast axis (α) of the linear birefringence property of the biological sample and the rotation angle (R) of the circular dichroism property can be calculated by equation (2) and equation (3) respectively.

$$α = \{ \begin{matrix}\frac{1}{2} arccos ( \frac{{S}_{0 °} ( 2 ) + {S}_{90 °} ( 2 )}{{S}_{0 °} ( 3 ) + {S}_{90 °} ( 3 )} ) & 0 ° < β < 90 ° \\ 0 & 90 ° < β < 180 °\end{matrix} ; \qquad ( 2 )$$

$$R = \{ \begin{matrix}\frac{{S}_{0 °} ( 0 )}{{S}_{0 °} ( 3 )} \pm \sqrt{\frac{{S}_{0 °} ( 0 )}{{S}_{0 °} ( 3 )} - 1} , 0 ° < β < 90 ° \\ - \frac{{S}_{0 °} ( 0 )}{{S}_{0 °} ( 3 )} \pm \sqrt{\frac{{S}_{0 °} ( 0 )}{{S}_{0 °} ( 3 )} - 1} , 90 ° < β < 180 °\end{matrix} ; \qquad ( 3 )$$

where β is a phase retardance of the linear birefringence property, and S_{0°} and S_{90°} are the Stokes vectors of a linear polarization state of the reflected light at an angle of the main axis of 0° and 90° respectively.
[Method for Detecting a Concentration of a miRNA in a Biological Sample]

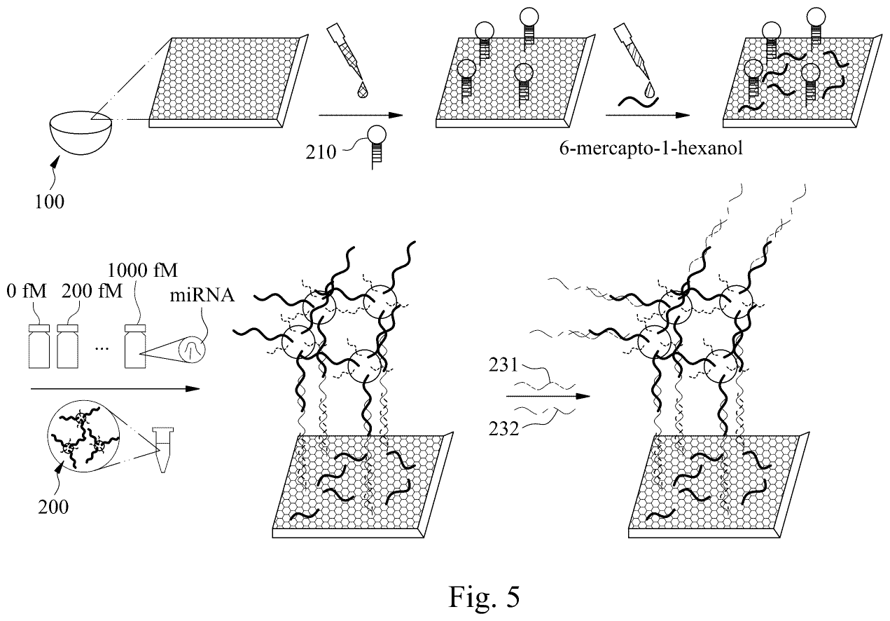

According to still another embodiment of the present disclosure, a method for detecting the concentration of the miRNA in the biological sample is provided. Reference is made to FIG. 4 and FIG. 5. FIG. 5 is a process schematic view showing the amplification of detection signals by the miRNA detection device 300 according to still another embodiment of the present disclosure. When using the dual-retarder polarimetry system 400 to detect miRNA, the detection probe 220 and the biological sample are mixed to form a mixture, then the mixture is added to the antimonene-based surface plasmon resonance prism coupler sensor 100 that has been seeded with the capture nucleic acid 210, and the reporter nucleic acid set 230 including the first reporter nucleic acid 231 and the second reporter nucleic acid 232 are added. The capture nucleic acid 210 binds to the miRNA in the biological sample and forms a complex with the detection probe 220 and the reporter nucleic acid set 230. Then, the light source 410 of the dual-retarder polarimetry system 400 is turned on to generate an incident light and incident on the complex along a light path. In detail, the incident light is incident on the polarizer 430 along the light path 460 to form a polarized light. The polarized light passes through the liquid crystal phase variable retarder group 440 to form a modulated polarized light. The modulated polarized light is incident on the miRNA detection device 300 and passes through the complex to form a reflected light. An optical information acquisition step is performed, in which the Stokes polarimeter 450 receives the reflected light formed after passing through the complex and generates optical information. Finally, a calculation step is performed, in which the computing module 480 receives the optical information generated by the Stokes polarimeter, calculates a decomposition Mueller matrix corresponding based on the optical information, and then calculates the principal angle of the fast axis (α) of the linear birefringence property and the rotation angle (R) of the circular dichroism property of the biological sample to detect the concentration of miRNA of the biological sample.

In the experiment, the antimonene-based surface plasmon resonance prism coupler sensor 100 of Example 1 was used for testing. First, the capture nucleic acid 210 containing the thiol group SH—C₆H₁₂— at the 5′ end (the sequence is referenced as SEQ ID NO: 1 and the sequence is referenced as SEQ ID NO: 3) was seeded on the antimonene layer 140 of Example 1, respectively. The capture nucleic acid 210 with the sequence referenced as SEQ ID NO: 1 can specifically bind to has-miR-125b-5p with the sequence referenced as SEQ ID NO: 2 (hereinafter referred as miR-125), and the capture nucleic acid 210 with the sequence referenced as SEQ ID NO: 3 can specifically bind to has-miR-21-5p with the sequence referenced as SEQ ID NO: 4 (hereinafter referred as miR-21), in which miR-125 and miR-21 can inhibit the proliferation and metastasis of cancer cells, and the related cancers are prostate cancer and breast cancer respectively.

The surface of Example 1 was first exposed to 10 μM of the capture nucleic acid 210 (dissolved in 1 M NaCl) for 2 hours. Then the surface of Example 1 was washed thoroughly with 10 mM PBS solution at pH 7.4. To decrease the likelihood of non-specific binding of nucleic acids to the surface of the gold thin film layer 130, the surface of Example 1 was passivated using 1 mM of 6-mercapto-1-hexanol (MCH) solution for 30 minutes at room temperature. Notably, the MCH not only assisted in suppressing nonspecific binding but also eliminated the capture nucleic acid 210 with thiol modification from the surface of Example 1. The first assistant nucleic acid 222 and the second assistant nucleic acid 223 containing the thiol group SH—C₆H₁₂— at 5′ end were also connected to the gold nanoparticle 221 to prepare the detection probe 220. The sequence of the first assistant nucleic acid 222 is referenced as SEQ ID NO: 5, the sequence of the second assistant nucleic acid 223 is TTTTT. In addition, the first reporter nucleic acid 231 with the sequence referenced as SEQ ID NO: 6 and the second reporter nucleic acid 232 with the sequence referenced as SEQ ID NO: 7 were added during detection.

When performing miRNA detection, miR-125 and miR-21 were first serially diluted to concentrations of 0 fM, 200 fM, 400 fM, 600 fM, 800 fM and 1000 fM respectively. Then 1 mL of the detection probe 220, 1 ml of miRNA (miR-125 or miR-21), 0.5 mL of first reporter nucleic acid 231 and 0.5 mL of second reporter nucleic acid 232 were sequentially added into the test tube and left to incubate for 15 minutes. Finally, measurement can be started to obtain optical information and then calculate the principal angle of the fast axis of the linear birefringence property and the rotation angle of the circular dichroism property of the miR-125 or miR-21. The test sample was measured from low concentration to high concentration. Before measuring the test sample of the next concentration, the test tube was rinsed once with PBS, and then the above experimental steps were repeated to measure the test sample of the next concentration.

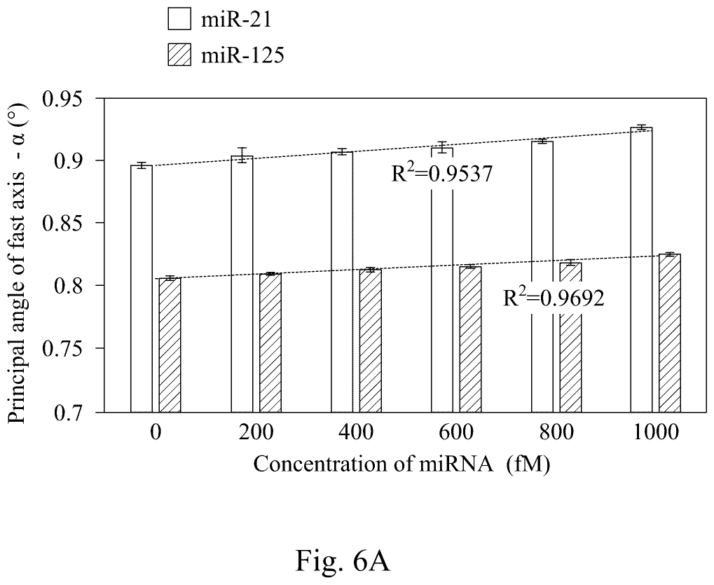

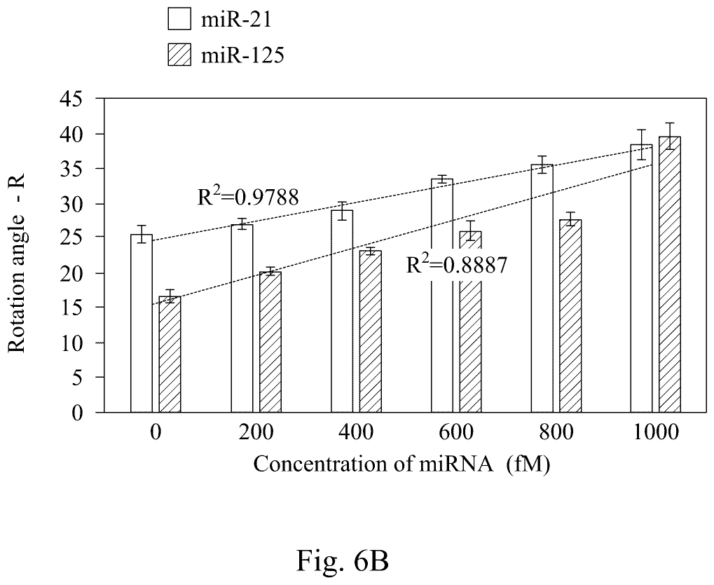

Reference is made to FIG. 6A and FIG. 6B. FIG. 6A shows a relationship between the principal angle of the fast axis of the linear birefringence property of miRNA (miR-125 and miR-21) and the concentration of miRNA, and FIG. 6B shows a relationship between the rotation angle of the circular dichroism property of miRNA (miR-125 and miR-21) and the concentration of miRNA. The results in FIG. 6A and FIG. 6B show that the principal angle of the fast axis (α) of the linear birefringence property and the rotation angle (R) of the circular dichroism property increase linearly with the miRNA concentration for both miR-125 and miR-21. The linear correlation coefficients (R²) vary in the range of 0.8887 to 0.9788, indicating that both the linear birefringence property and the circular dichroism property provide a reliable method of predicting the concentrations of miR-125 and miR-21. The results in FIG. 6A show that the sensitivity and the resolution of the dual-retarder polarimetry system 400 of the present disclosure are 2.53×10^{-5o}/(fM) and 76.91 fM, respectively, when taking the principal angle of the fast axis (α) of the linear birefringence property for evaluation purposes. The results in FIG. 6B show that the sensitivity and the resolution of the dual-retarder polarimetry system 400 of the present disclosure are 17.95×10⁻⁵ R/(fM) and 69.41 fM, respectively, when using the rotation angle (R) of the circular dichroism property for calculation purposes. Generally speaking, the average standard deviation values of the principal angle of the fast axis (α) and the rotation angle (R) measured by a surface plasmon resonance sensor are 2.04×10^{-3o} and 1.125 R, respectively. Notably, the values of the principal angle of the fast axis (α) and the rotation angle (R) obtained by detecting miR-125 are different from those obtained by detecting miR-21 with the same concentration. The results confirm that the antimonene-based surface plasmon resonance prism coupler sensor 100, the miRNA detection device 300 and the dual-retarder polarimetry system 400 have good selectivity for different types of miRNA.

In summary, the antimonene-based surface plasmon resonance prism coupler sensor is used with the nucleic acid-linked gold nanoparticle to amplify detection signals, and used with the decomposition Mueller matrix to detect the linear birefringence property and the circular dichroism property of miRNA at concentrations from 0 fM to 1000 fM in the present disclosure. The concentration of miRNA is determined based on the pre-established calibration curve, and the changes in the linear birefringence property and the circular dichroism property of the miRNA are linearly related to the changes in the miRNA concentration. The values of the linear birefringence property and the circular dichroism property detected for different biological samples are different, which proves that the antimonene-based surface plasmon resonance prism coupler sensor, the miRNA detection device and the dual-retarder polarimetry system of the present disclosure can rapidly and selectively detect the presence and concentration of miRNA in the biological sample. Therefore, the antimonene-based surface plasmon resonance prism coupler sensor, the miRNA detection device and the dual-retarder polarimetry system of the present disclosure have the potential to serve as a valuable tool for miRNA detection with prospective applications in cancer diagnosis.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An antimonene-based surface plasmon resonance prism coupler sensor, comprising:
a prism having a surface;
a tantalum pentoxide thin film layer disposed on the surface of the prism;
a gold thin film layer disposed on the tantalum pentoxide thin film layer; and
an antimonene layer disposed on the gold thin film layer.

2. The antimonene-based surface plasmon resonance prism coupler sensor of claim 1, wherein a thickness of the gold thin film layer is greater than a thickness of the tantalum pentoxide thin film layer and a thickness of the antimonene layer.

3. The antimonene-based surface plasmon resonance prism coupler sensor of claim 1, wherein the antimonene layer is composed of a plurality of layers of antimonene.

4. A miRNA detection device, which is for detecting a miRNA in a biological sample, comprising:
the antimonene-based surface plasmon resonance prism coupler sensor of claim 1;
a capture nucleic acid seeded on the antimonene layer of the antimonene-based surface plasmon resonance prism coupler sensor, wherein the capture nucleic acid specifically binds to the miRNA;
a detection probe for amplifying detection signals, wherein the detection probe comprises a gold nanoparticle, a first assistant nucleic acid and a second assistant nucleic acid, bases at one end of the first assistant nucleic acid and bases at one end of the second assistant nucleic acid are respectively connected to the gold nanoparticle, and the first assistant nucleic acid specifically binds to the capture nucleic acid; and
a reporter nucleic acid set comprising a first reporter nucleic acid and a second reporter nucleic acid, wherein the first reporter nucleic acid specifically binds to the capture nucleic acid, and the second reporter nucleic acid specifically binds to the capture nucleic acid and the first assistant nucleic acid.

5. A dual-retarder polarimetry system, comprising:
the miRNA detection device of claim 4, which is in contact with a biological sample;
a light source configured to generate an incident light, wherein the incident light is incident on the miRNA detection device along a light path;
a polarizer disposed between the light source and the miRNA detection device, and configured to polarize the incident light into a polarized light;
a liquid crystal phase variable retarder group disposed between the polarizer and the miRNA detection device and configured to receive the polarized light and change polarization states of the polarized light to form a modulated polarized light, wherein the liquid crystal variable retarder group comprises:
a first liquid crystal phase variable retarder with an angle of a main axis at 90°; and
a second liquid crystal phase variable retarder with an angle of the main axis at 45°, wherein the second liquid crystal phase variable retarder is closer to the miRNA detection device than the first liquid crystal phase variable retarder;
a Stokes polarimeter configured to receive a reflected light formed by the modulated polarized light after passing through the miRNA detection device and generate optical information; and
a computing module configured to receive the optical information generated by the Stokes polarimeter, calculate a decomposition Muller matrix corresponding, and then calculate a linear birefringence property and a circular dichroism property of the biological sample, so as to detect a concentration of a miRNA in the biological sample.

6. The dual-retarder polarimetry system of claim 5, wherein the decomposition Muller matrix is expressed as equation (1):
$${M}_{sample} = {M}_{l b} {M}_{c d} {M}_{R} {M}_{D} \qquad ( 1 )$$
where Mₛₐₘₚₗₑ is a Muller matrix of the biological sample, M_{lb} is a Muller matrix of the linear birefringence property of the biological sample, M_{cd} is a Muller matrix of the circular dichroism property of the biological sample, M_{R} is a Mueller matrix of a reflectance of the miRNA detection device, and M_{D} is a Mueller matrix of a depolarization effect of the biological sample.

7. The dual-retarder polarimetry system of claim 6, wherein the linear birefringence property is calculated as a principal angle of a fast axis (α), and the principal angle of the fast axis (α) is calculated by equation (2):
$$α = \{ \begin{matrix}\frac{1}{2} arccos ( \frac{{S}_{0 °} ( 2 ) + {S}_{90 °} ( 2 )}{{S}_{0 °} ( 3 ) + {S}_{90 °} ( 3 )} ) & 0 ° < β < 90 ° \\ 0 & 90 ° < β < 180 °\end{matrix} ; \qquad ( 2 )$$
where β is a phase retardance of the linear birefringence property, and S_{0°} and S_{90°} are a Stokes vector of a linear polarization state of the reflected light at an angle of the main axis of 0° and 90° respectively.

8. The dual-retarder polarimetry system of claim 7, wherein the circular dichroism property is calculated as a rotation angle (R), and the rotation angle (R) is calculated by equation (3):
$$R = \{ \begin{matrix}\frac{{S}_{0 °} ( 0 )}{{S}_{0 °} ( 3 )} \pm \sqrt{\frac{{S}_{0 °} ( 0 )}{{S}_{0 °} ( 3 )} - 1} , 0 ° < β < 90 ° \\ - \frac{{S}_{0 °} ( 0 )}{{S}_{0 °} ( 3 )} \pm \sqrt{\frac{{S}_{0 °} ( 0 )}{{S}_{0 °} ( 3 )} - 1} , 90 ° < β < 180 °\end{matrix} ; \qquad ( 3 )$$
where β is the phase retardance of the linear birefringence property, and S_{0°} is the Stokes vector of the linear polarization state of the reflected light at the angle of the main axis of 0°.

9. A method for detecting a concentration of a miRNA in a biological sample, comprising:
providing the dual-retarder polarimetry system of claim 7;
mixing the biological sample with the detection probe to form a mixture;
adding the mixture and the reporter nucleic acid set to the antimonene-based surface plasmon resonance prism coupler sensor, wherein the capture nucleic acid binds to the miRNA in the biological sample and forms a complex with the detection probe and the reporter nucleic acid set;
turning on the light source to generate an incident light and incident on the complex along a light path;
performing an optical information acquisition step, wherein the Stokes polarimeter receives a reflected light formed after passing through the complex and generates optical information; and
performing a calculation step, wherein the computing module receives the optical information generated by the Stokes polarimeter, calculates a decomposition Muller matrix corresponding, and then calculates a linear birefringence property and a circular dichroism property of the biological sample, so as to detect the concentration of the miRNA in the biological sample.

10. The method of claim 9, wherein a detection range of the dual-retarder polarimetry system is from 0 fM to 1000 fM.
